(19) United States
(12) Patent Application Publication
(10) Pub. No.: US 2024/0282464 A1
(43) Pub. Date: Aug. 22, 2024

(54) SYSTEM AND METHOD FOR DETERMING GEMETIC POPULATION COMPOSITION USING HYBRID SPECIFIC REFERENCE GENETIC DATA GENERATION FOR POPULATION, BREED, DISEASE GROUPS, AND SPECIES AND ANALYSIS FOR DETERMINIG GENETIC COMPONENTS

(71) Applicant: CLINOMICS INC., Ulsan (KR)

(72) Inventor: Sung Bong KANG, Suwon-si (KR)

(21) Appl. No.: 18/024,969

(22) PCT Filed: Nov. 16, 2022
(86) PCT No.: PCT/KR2022/018119

§ 371 Date: May 13, 2023

(30) Foreign Application Priority Data
Nov. 19, 2021 (KR) 10-2021-0160791

(51) Int. Cl. G16H 50/80; G16H 50/70

(52) U.S. Cl. CPC G16H 50/80; G16H 50/70

(57) ABSTRACT

The present invention relates to a system and method for generating specific reference genetic data of mixture of population and disease population or breed or hybrid and determining genetic population composition, and the-be-solved task is to create a genome representing the population, create a hybrid representative through a crossbreeding simulation between the representatives, measure the genetic similarity between the new data between the representative of the population and the representative of the hybrid, and determine the composition of the population, thereby determining the genetic population composition of test target individuals. As an example, provided is a system for determining genetic population composition comprising a population representative individual selection unit for measuring the frequency of occurrence of preselected genotypes for individuals in homogenous populations and selecting a population representative individuals for the respective homogenous populations according to the measured frequency of occurrence, and a genetic population composition determination unit for generating hybrid data of the population representative individuals for the respective generation through repetitive hybridization between the population representative individuals and determining the genetic population composition of the test target individual according to the genetic similarity between the hybrid data and the test target individual.

### TECHNICAL FIELD

An embodiment of the present invention relates to a system and method for generating specific reference genetic data of mixture of population and disease population or breed or hybrid and determining genetic population composition.

### BACKGROUND ART

It is well known to create a standard genome that is representative of a particular population. In the past, many countries have invested heavily in creating a standard genome specific to a dog breed, or in recent years, creating a standard genome specific to that country.

According to most perceptions, it is easy to obtain genetic makeup information for hybrids by creating a population reference genome and using painting techniques. For example, a 1:1 hybrid of populations A and B would exactly match the standard genome of each population, 50% A and 50% B. However, these methods are not accurate. That is, when a specific single nucleotide polymorphism (SNP) in population A has only AA and the SNP at the same site has only GG in population B, the A-B hybrid has the AG genotype. However, if there is no case where both populations A and B have AG, it can be determined as population C that can have AG, so genetic information on hybrids is required in advance.

Meanwhile, the conventional ancestry analysis method finds a specific genotype or pattern of population and uses the ‘chromosome painting’ technique to discriminate the population by having the genotype or searches for genetic origin through information on the maternal mitochondria (MT) and paternal Y genomes.

Further, Mendel's laws of inheritance, which explain the genetic principles for conventional ancestry analysis, are well known as the laws that Gregor Mendel (1822-1884) experimented with peas in 1865, summarized how genetic factors are inherited and phenotypes appear, and interpreted probabilistically.

Prior art related to the present invention includes US Patent Publication US2017-0004256A1, US Patent Publication US2017-0017757A1, US Patent Publication US2017-0199959A1, U.S. Pat. No. 8,620,594B2, European Publication Patent EP3588506A1, PCT International Publication No. WO2017-210542A1, US Patent Publication US2008-0255768A1, Korean Patent Registration No. 10-2138165, and Korean Patent Publication No. 10-2021-0089073.

### DISCLOSURE

### Technical Problem

Embodiments of the present invention provide a system and method for determining genetic population composition using specific reference genome data of populations and hybrids, which creates a genome representative of a population, creates hybrid representatives through crossbreeding simulations between representatives, measures genetic similarity between new data about the population representatives and hybrid representatives, and determines the population composition, thereby determining the genetic population composition of the to-be tested individual.

### Technical Solution

A system for generating specific reference genetic data of mixture of population and disease population or breed or hybrid and determining genetic population composition according to an embodiment of the present invention comprises a population representative individual selection unit for measuring the frequency of occurrence of preselected genotypes for individuals in homogenous populations and selecting a population representative individuals for the respective homogenous populations according to the measured frequency of occurrence, and a genetic population composition determination unit for generating hybrid data of the population representative individuals for the respective generation through repetitive hybridization between the population representative individuals and determining the genetic population composition of the test target individual according to the genetic similarity between the hybrid data and the test target individual.

Further, the population representative individual selection unit may comprise a genome data collection unit for collecting genomic data for the respective populations, a homogenous population classification unit for measuring genetic similarity between populations using the genetic data and classifying into homogenous populations according to the measurement result, and a population representative individual genome generation unit for measuring the frequency of occurrence of a preselected genotype at the same genetic locus among individuals in the homogenous population and selecting population representative individuals for the respective homogenous populations according to the measured frequency of occurrence to generate a genome for the selected population representative individuals.

Further, the homogenous population classification unit may remove individuals that do not cluster into homogenous populations.

Further, the population representative individual generation unit may select an individual with the highest frequency of occurrence as the population representative individual, and the population representative individual is randomly selected from two or more individuals having the genotype with the same frequency of occurrence.

Further, the population representative individual generation unit may remove the individual when the frequency of occurrence is less than or equal to a predetermined reference frequency.

Further, the population representative genome generation unit may measure the genetic similarity between population representative individuals in the same generation and select, if the similarity is higher than a predetermined reference level, the corresponding population representative individual as one common population representative individual.

Further, the genetic population composition determination unit may comprise a hybrid data generation unit for generating hybrid data of the population representative individuals for the respective generations through repetitive hybridization between the population representative individuals and a test target individual breed determination unit for measuring the genetic similarity between the hybrid data and the test target individual and determining the breed of the test target individual according to the measurement result.

Further, the hybrid data generation unit may determine a combination according to the formula (Equation, #Representator) at the time of repeated hybridization between the first, second, third, and higher generation population representative individuals,

| Generation | Equation | #Representator |
|---|---|---|
| 1^{st} | N | N |
| 2^{nd} | (n + 2 − 1)!/{(n − 1)!*2!} | (N − 1)!/2(n − 1)! − #(1^{st}) |
| 3^{rd} | (n + 4 − 1)!/{(n − 1)!*4!)} | (N + 3)!/24(N − 1)! − |
| | | #(2^{nd}) − #(1^{st}) |
| . . . | . . . | . . . |
| m | (n + 2\hat{ }(m − 1) − 1)!/{(n − | (N + 2\hat{ }(m − 1) − 1)!/{(N − |
| generation | 1)!*2\hat{ }(m − 1)!} | 1)!*2(m − 1)!} − Σ₁^{m-1} |
| | | # generations |

the Equation may represent the total number of population representative individuals that m generation has without considering previous generations, the #Representator may represent the total number of population representative individuals used in the respective generations, and N of the Equation and #Representator may be the number of populations.

Further, the test target individual breed determination unit may presume that the genetic population composition of the population representative individual corresponding to the hybrid data having the highest genetic similarity with the test target individual among the hybrid data is the genetic population composition of the test target individual.

Further, the test target individual breed determination unit may sort the population representative individuals in the order of high genetic similarity with the test target individual, converts the genetic similarity of the respective sorted population representative individuals into a percentage, and divide the converted percentage value by the proportion of the respective population representatives in the total population representatives to estimate the divided value as an approximation of a positive integer, thereby identifying the genetic population composition of the test target individual of the next generation, not a specific generation.

A method for generating specific standard genetic data of mixture of population and disease population or breed or hybrid and determining genetic population composition according to another embodiment of the present invention comprises a population representative individual selecting step for measuring the frequency of occurrence of preselected genotypes for individuals in homogenous populations and selecting a population representative individuals for the respective homogenous populations according to the measured frequency of occurrence, and a genetic population composition determining step for generating hybrid data of the population representative individuals for the respective generation through repetitive hybridization between the population representative individuals and determining the genetic population composition of the test target individual according to the genetic similarity between the hybrid data and the test target individual.

Further, the population representative individual selection unit may comprise a genome data collection unit for collecting genomic data for the respective populations, a homogenous population classification unit for measuring genetic similarity between populations using the genetic data and classifying into homogenous populations according to the measurement result, and a population representative individual genome generation unit for measuring the frequency of occurrence of a preselected genotype at the same genetic locus among individuals in the homogenous population and selecting population representative individuals for the respective homogenous populations according to the measured frequency of occurrence to generate a genome for the selected population representative individuals.

Further, the homogenous population classifying step may remove the individual that does not cluster into homogenous populations.

Further, the population representative individual generating step may select an individual with the highest frequency of occurrence as the population representative individual, and the population representative individual is randomly selected from two or more individuals having the genotype with same frequency of occurrence.

Further, the population representative individual generating step may remove the individual when the frequency of occurrence is less than or equal to a predetermined reference frequency.

Further, the population representative genome generating step may measure the genetic similarity between population representative individuals in the same generation and select, if the similarity is higher than a predetermined reference level, the corresponding population representative individual as one common population representative individual.

Further, the genetic population composition determining step may comprise a hybrid data generating step for generating hybrid data of the population representative individuals for the respective generations through repetitive hybridization between the population representative individuals, and a test target individual breed determining step for measuring the genetic similarity between the hybrid data and the test target individual and determining the breed of the test target individual according to the measurement result.

Further, the hybrid data generating step may determine a combination according to the formula (Equation, #Representator) at the time of repeated hybridization between the first, second, third, and higher generation population representative individuals,

| Generation | Equation | #Representator |
|---|---|---|
| 1^{st} | N | N |
| 2^{nd} | (n + 2 − 1)!/{(n − 1)!*2!} | (N − 1)!/2(n − 1)! − #(1^{st}) |
| 3^{rd} | (n + 4 − 1)!/{(n − 1)!*4!)} | (N + 3)!/24(N − 1)! − |
| | | #(2^{nd}) − #(1^{st}) |
| . . . | . . . | . . . |
| m | (n + 2\hat{ }(m − 1) − 1)!/{(n − | (N + 2\hat{ }(m − 1) − 1)!/{(N − |
| generation | 1)!*2\hat{ }(m − 1)!} | 1)!*2(m − 1)!} − Σ₁^{m-1} |
| | | # generations |

the Equation may represent the total number of population representative individuals that m generation has without considering previous generations, the #Representator may represent the total number of population representative individuals used in the respective generations, and N of the Equation and #Representator may be the number of populations.

Further, the test target individual breed determining step may presume that the genetic population composition of the population representative individual corresponding to the hybrid data having the highest genetic similarity with the test target individual among the hybrid data is the genetic population composition of the test target individual.

Further, the test target individual breed determination unit may sort the population representative individuals in the order of high genetic similarity with the test target individual, converts the genetic similarity of the respective sorted population representative individuals into a percentage, and divide the converted percentage value by the proportion of the respective population representatives in the total population representatives to estimate the divided value as an approximation of a positive integer, thereby identifying the genetic population composition of the test target individual of the next generation, not a specific generation.

### Advantageous Effects

According to the present invention, it may provide a system and method for determining genetic population composition using specific reference genome data of populations and hybrids, which creates a genome representative of a population, creates hybrid representatives through crossbreeding simulations between representatives, measures genetic similarity between new data about the population representatives and hybrid representatives, and determines the population composition, thereby determining the genetic population composition of the to-be tested individual.

Although the present invention has been described below based on genotype, the same principle can be applied to population representative composition and hybridization analysis based on population representative haplotype.

Further, examples and preliminary analysis of the generation of representative individuals of the present invention refer to individuals generated through genotype voting, as well as virtual generated through simulation.

Further, the term “population” may be used for any population that can be divided into a cat, human or other pets or plants, even disease populations, as an example of a disease population, when a representative genome of a very sophisticated lung cancer population was generated, in order to determine the risk of lung cancer of a specific individual, the risk of germline lung cancer can be evaluated by mapping to the corresponding representative genome and identifying the mapping rate. Further, through hybridization of lung cancer and gastric cancer representatives, the lung cancer genetic risk, gastric cancer genetic risk, overall genetic risk, etc. of a specific individual can be evaluated by generating a lung cancer-stomach cancer hybrid representative. In the age of the inverse population pyramid structure, as interest, demand, and research on wellness increase, the new approach of this technology is expected to provide a new perspective to understanding the relationship between diseases and disease populations.

### DESCRIPTION OF DRAWINGS

FIG. 1 is a block diagram showing the overall configuration of a system for determining a genetic population composition using specific reference genome data of populations and hybrids according to an embodiment of the present invention.

FIG. 2 is a view showing an example of the execution result of a homogenous population classification unit that determines an heterogeneous individual and a homogenous population through the measurement of genetic similarity between individuals according to an embodiment of the present invention.

FIG. 3 is a view showing an example of an execution result of a population representative individual genome generation unit generating a population representative genome through measurement of the frequency of genotype occurrence according to an embodiment of the present invention.

FIG. 4 is a schematic view showing an example of generating a new hybrid through hybridization between genomes of representative individuals of a population based on Mendel's laws of inheritance by the hybrid data generation unit according to an embodiment of the present invention.

FIG. 5 is a view showing how representative individuals of the previous generation are used in the next generation when generations are repeated according to an embodiment of the present invention.

FIG. 6 is a diagram for explaining the ratio of the genetic population composition and the method for determining the population composition of the third-generation through analysis up to the second-generation according to an embodiment of the present invention.

FIG. 7 is a view showing example data for confirming population composition through pattern analysis for ‘Akita’ and ‘Chow-Chow’ hybrids according to an embodiment of the present invention.

FIG. 8 is a flow chart showing the overall configuration of a method for determining a genetic population composition using specific reference genome data of populations and hybrids according to an embodiment of the present invention.

### MODE FOR INVENTION

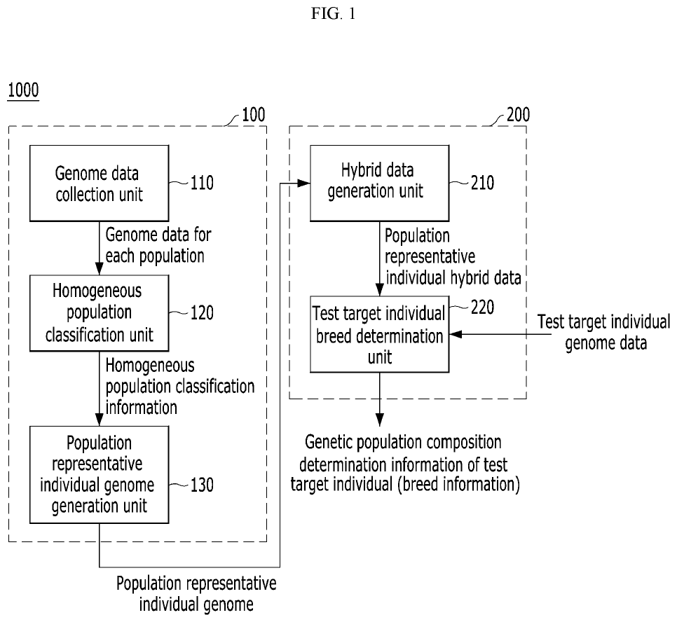

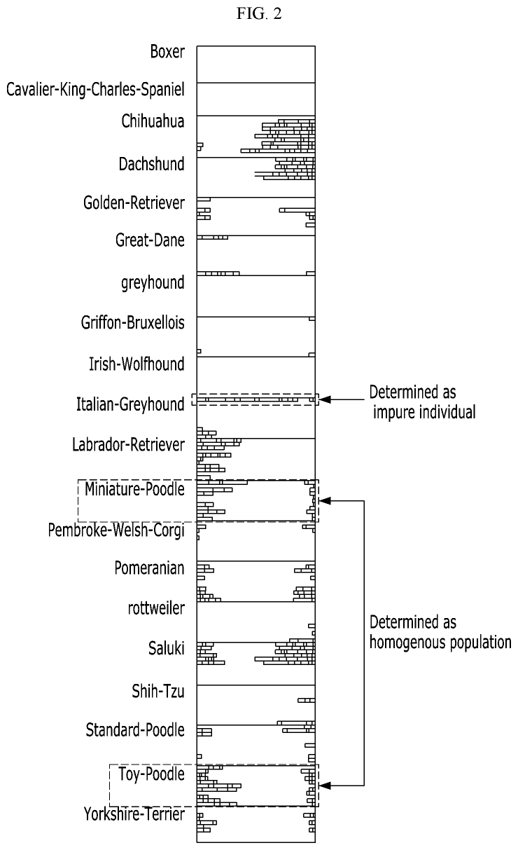

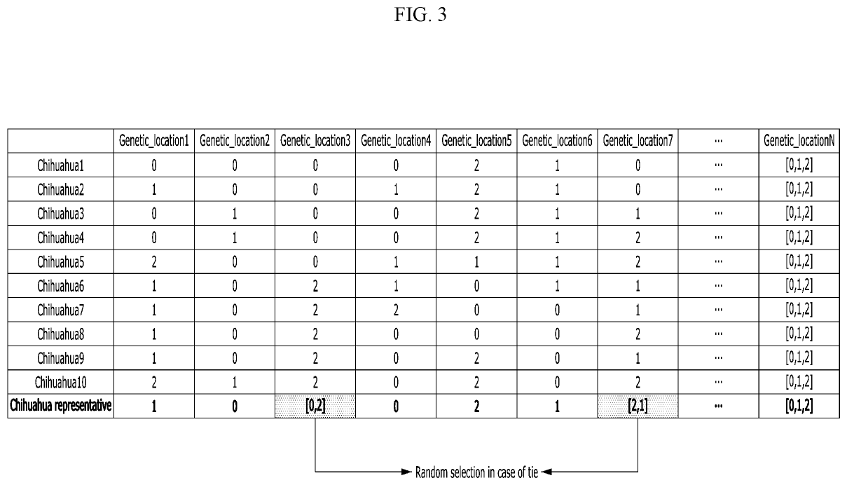

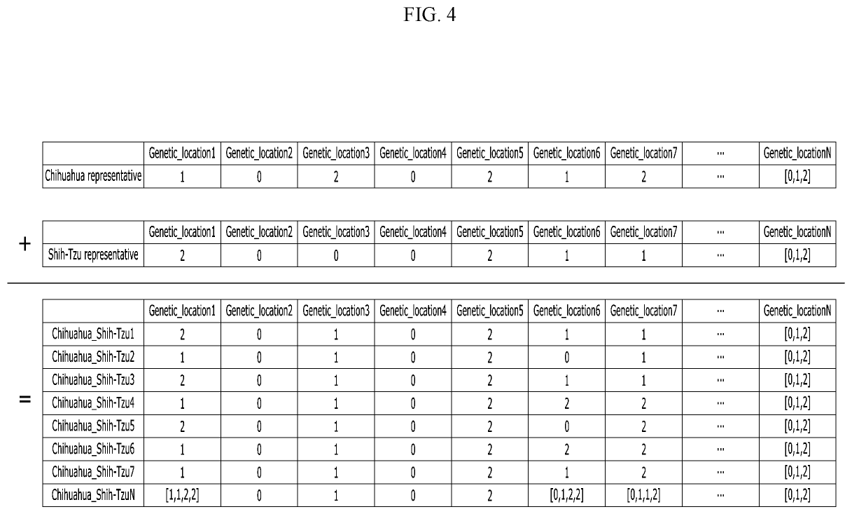

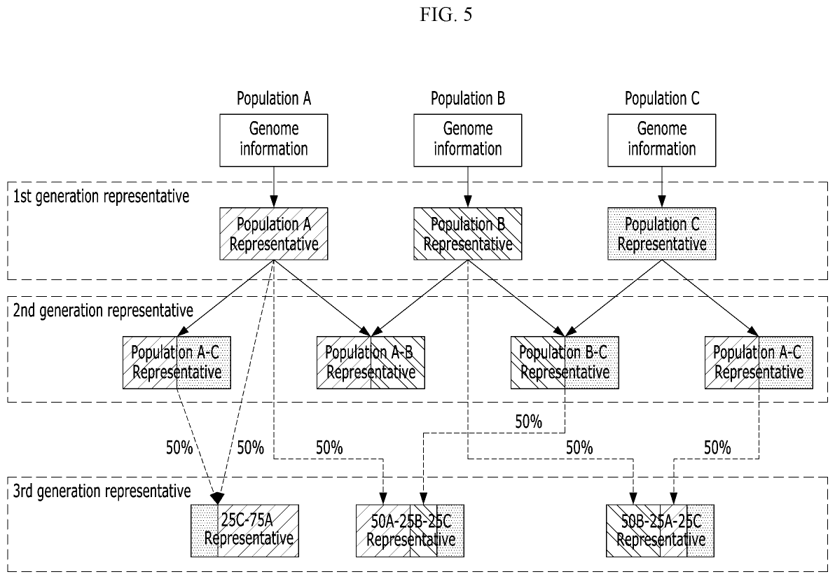

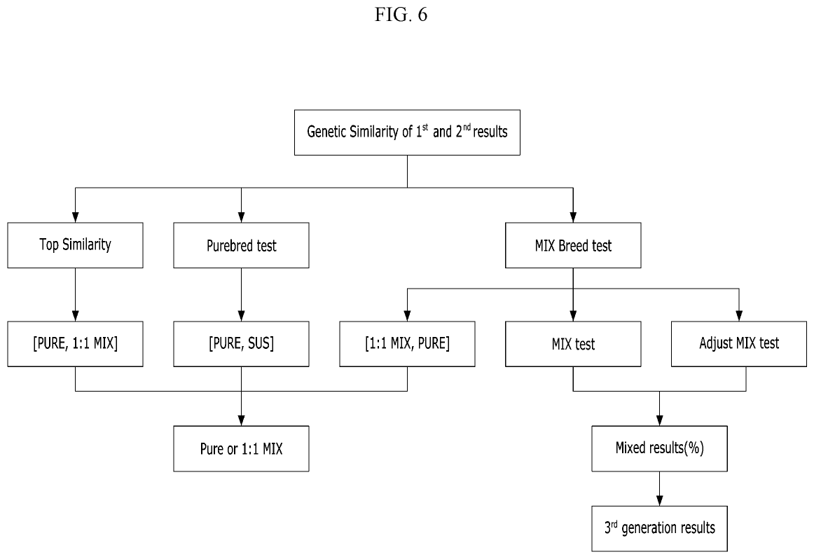

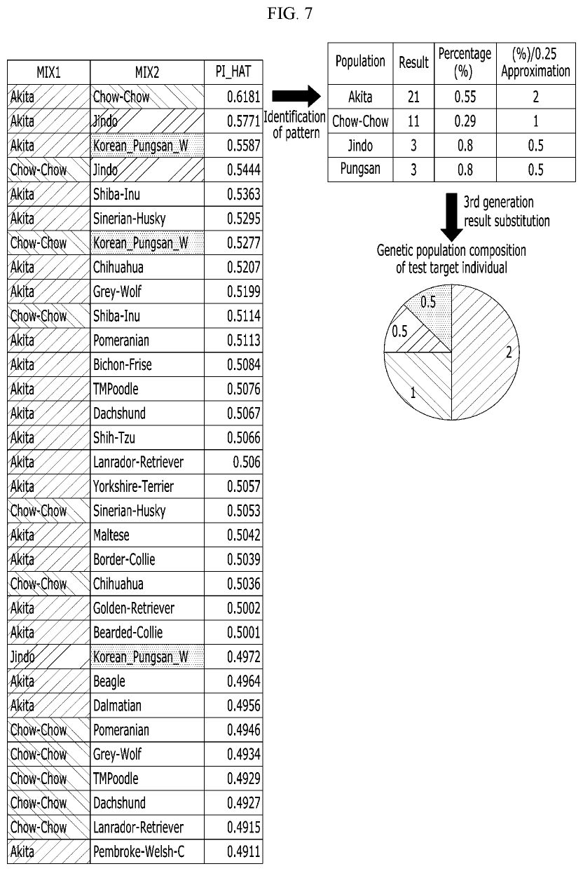

FIG. 1 is a block diagram showing the overall configuration of a system for determining a genetic population composition using specific reference genome data of populations and hybrids according to an embodiment of the present invention, FIG. 2 is a view showing an example of the execution result of a homogenous population classification unit that determines an heterogeneous individual and a homogenous population through the measurement of genetic similarity between individuals according to an embodiment of the present invention, FIG. 3 is a view showing an example of an execution result of a population representative individual genome generation unit generating a population representative genome through measurement of the frequency of genotype occurrence according to an embodiment of the present invention, FIG. 4 is a schematic view showing an example of generating a new hybrid through hybridization between genomes of representative individuals of a population (crossing between individuals) based on Mendel's laws of inheritance by the hybrid data generation unit according to an embodiment of the present invention, FIG. 5 is a view showing how representative individuals of the previous generation are used in the next generation when generations are repeated according to an embodiment of the present invention, FIG. 6 is a diagram for explaining the ratio of the genetic population composition and the method for determining the population composition of the third-generation through analysis up to the second-generation according to an embodiment of the present invention, and FIG. 7 is a view showing example data for confirming population composition through pattern analysis for ‘Akita’ and ‘Chow-Chow’ hybrids according to an embodiment of the present invention.

Referring to FIG. 1, the system for determining a genetic population composition using specific reference genome data of populations and hybrids 1000 according to an embodiment of the present invention may comprise at least one of a population representative individual selection unit 100 and a genetic population composition determination unit 200.

The population representative individual selection unit 100 may measure the frequency of occurrence of a pre-selected genotype for individuals in the homogenous population and select a population representative individual for each of the homogenous populations according to the measured frequency of occurrence.

To this end, as shown in FIG. 1, the population representative individual selection unit 100 may include at least one of a genome data collection unit 110, a homogenous population classification unit 120, and a population representative individual genome generation unit 130.

The genome data collection unit 110 may collect a large amount of genome data (sanger, NSG, micro-array, etc.) for each population (e.g., topographical and phenotypical populations) and store and manage the collected genome data for each population.

The homogenous population classification unit 120 may measure the genetic similarity between populations using a large amount of genome data collected through the genome data collection unit 110 and cluster and classify into homogenous populations according to the measurement result. The homogenous population classification unit 120 may cluster populations that can be classified as homogenous populations according to the degree of similarity for each population into homogenous populations and accumulate data accordingly. The homogenous population classification unit 120 may apply a method for measuring genetic similarity between individuals in a population, such as the ‘Admixture’ method shown in FIG. 2 and may not only identify and merge the same population, but also eliminate other individuals who are not members of the population through this method. If they are otherwise collected from different sources, they may be treated as different names of populations.

The population representative individual genome generation unit 130 may measure the frequency of occurrence of a pre-selected genotype for each identical genetic location among individuals in the homogenous population and select the frequency of occurrence of a pre-selected genotype and select a population representative individual for each homogenous population according to the measured frequency of occurrence to create genomes for representative individuals of the population.

More specifically, the population representative individual genome generation unit 130 may select the individual with the highest frequency of occurrence as the population representative individual of the first-generation in which a population representative individual may be selected in a random manner for two or more individuals having genotypes having the same frequency of occurrence. That is, in the process of generating a population representative through the measurement of the frequency of occurrence of a genotype for each genetic location among individuals in a population, for example, as shown in FIG. 3, a population representative may be randomly selected in the case of the same frequency of occurrence of genotype.

Further, when the frequency of occurrence is equal to or less than a predetermined reference frequency, the population representative individual genome generation unit 130 may remove the corresponding individual. If there are heterogeneous individuals that have not been filtered through the homogenous population classification unit 120 by measuring the frequency of occurrence of the genotype, the genotype of the heterogeneous individual may be removed through the measurement of the frequency of occurrence of the genotype. For example, when a population consisting of 100 Koreans is collected, if one Japanese is not filtered out through the homogenous population classification unit 120, through the measurement of the frequency of occurrence of genotypes, genotypes commonly possessed by 99 Koreans are adopted, thereby minimizing the effect of one Japanese to the fullest.

Further, the population representative genome generation unit 130 may measure the genetic similarity between population representative individuals in the same generation and select, if the similarity is higher than a predetermined cutoff level, the corresponding population representative individual as one common population representative individual. When the representative individual of the population first created in this embodiment is referred to as the first-generation population representative individual, the first-generation population representative individual refers to a collection of information about the structure and genotype of the genome that is frequently occurred in the population. If the first-generation representative of population A and the first-generation representative of population B are genetically very close, characteristics such as origin, traffic, common ancestry, and phenotype between populations A and B are identified to create common first-generation population representative.

The genetic population composition determination unit 200 generates hybrid data of the population representative individual for each generation through repetitive hybridization between the population representative individuals and determines the genetic population composition of the test target individual according to the genetic similarity between the hybrid data and the test target individual.

To this end, the genetic population composition determination unit 200 may comprise at least one of a hybrid data generation unit 210 and a test target individual breed determination unit 220, as shown in FIG. 1.

The hybrid data generation unit 210 may generate hybrid data of population representative individuals for each generation through repetitive hybridization between population representative individuals. FIG. 4 shows an example of a hybridization process, in which the genotype is determined according to Mendel's laws of inheritance in the hybridization process. The generated 1:1 hybrid is referred to as the second-generation, and genotype voting is performed on newly generated second-generation individuals to generate second-generation representative individuals as shown in FIG. 3. One second-generation representative individual includes the genetic information of the first-generation representative individual of the two populations in a 50:50 ratio. In this way, hybrid data for a 3rd generation individual can be created using the genetic information of the second-generation representative individual and the second-generation representative individual.

The hybrid data for the representative individuals of the respective generations thus generated may also be used when generation representative individual data described later is generated. For example, as shown in FIG. 5, if population A, population B, and population C have a composition of 50:25:25, respectively, in a third-generation individual, ‘A:B:C=50:25:25’ individual may be created using a first-generation population A representative and a second-generation population B-C representative. As in the case of creating second-generation representative individuals, the third-generation individuals may be created through repeated hybridization, and the third-generation representatives may be created using these third-generation individuals.

By the hybrid data generation unit 210, the number of representatives up to the third-generation may be determined by Equation 1 below, which is a combination formula including duplication. That is, the combination in repetitive hybridization between population representative individuals according to the first-generation, second-generation, third-generation, and higher generations may be determined according to Equation 1 (Equation, #Representator) below.

[Equation 1]

| Generation | Equation | #Representator |
|---|---|---|
| 1^{st} | N | N |
| 2^{nd} | (n + 2 − 1)!/{(n − 1)!*2!} | (N − 1)!/2(n − 1)! − #(1^{st}) |
| 3^{rd} | (n + 4 − 1)!/{(n − 1)!*4!)} | (N + 3)!/24(N − 1)! − |
| | | #(2^{nd}) − #(1^{st}) |
| . . . | . . . | . . . |
| m | (n + 2\hat{ }(m − 1) − 1)!/{(n − | (N + 2\hat{ }(m − 1) − 1)!/{(N − |
| generation | 1)!*2\hat{ }(m − 1)!} | 1)!*2(m − 1)!} − Σ₁^{m-1} |
| | | # generations |

In Equation 1, Equation means the total number of population representative individuals of m generation without considering previous generations, #Representator means the total number of population representative individuals used in each generation, and N in Equation and #Representator means the number of populations. More specifically, Equation of Equation 1 represents the combination formula of populations including duplicates, n of Equation represents the number of populations to be identified, and m represents the number of generations. Equation represents the total number of population representations that may be had in m generation without considering the number of previous generations. N of #Representator represents the number of populations, and m represents the number of generations like Equation. Equation represents the total number of population representatives from each generation, and #Representator represents the number of population representatives directly used by each generation.

The test target individual breed determination unit 220 may measure the genetic similarity between the hybrid data and the test target individual generated by the hybrid data generation unit 210 and identify the breed of the test target individual (genetic population composition) according to the measurement result. More specifically, the test target individual breed determination unit 220 may measure the genetic similarity with the test target individual among the hybrid data to assume the genetic population composition of the population representative individual corresponding to the hybrid data with the highest degree of similarity to the genetic population composition of the test target individual. In this way, which population is closest to a particular generation can be identified by comparing new individuals of the particular generation with representatives of that generation and representatives of previous generations. For example, in order to determine which population is closest to the parent generation (second-generation), N's individual may be compared with the representatives of the first and second-generations to identify which representative is closest to ‘Identity-By-Descent.’ If it is closest to the representative A-B of the second-generation, the genetic population composition of N is represented by A-B, and if it is closest to the representative A of the first-generation, the population composition of N is represented by A-A. Although this embodiment describes the analysis method through the measurement of ‘Identity-By-Descent,’ it may include all other methods for measuring genetic similarity.

Further, the test target individual breed determination unit 220 may sort the population representative individuals in the order of high genetic similarity with the test target individual, converts the genetic similarity of the respective sorted population representative individuals into a percentage, and divide the converted percentage value by the proportion of the respective population representatives in the total population representatives to estimate the divided value as an approximation of a positive integer, thereby identifying the genetic population composition of the test target individual of the next generation, not a specific generation. The test target individual breed determination unit 220 may identify the next population or percentage through pattern analysis of genetic similarity results in order to confirm the percentage of the next generation and genetic population composition, not a specific generation. Here, pattern analysis may be performed by ensemble of several tests to confirm the result.

FIG. 6 shows a schematic view of how to predict the percentage of a population and the pedigree of a population in the next generation using the genetic similarity results for representatives of the first and second-generations. First of all, it is determined whether the input can be expressed with only the first and second-generations, and if it cannot be expressed with only the first and second-generations, as shown in FIG. 7, after sorting in order of similarity, a pattern for the result may be identified. The population according to the identified pattern is converted into a percentage and divided by the number of results in the next generation (the third-generation is the number of 4 representative individuals). As shown in FIG. 7, the results for ‘Akita,’ ‘Chow-Chow,’ ‘Jindo,’ and ‘Pungsan’ are converted into percentages (55%, 29%, 8%, and 8%), these values are divided by 0.25, i.e., each individual's share of the total population to estimate an approximate value (rounding method), thereby obtaining the third-generation result of ‘2, 1, 0.5, and 0.5,’ respectively, and the result may be estimated as the genetic population composition of the to-be-tested individual. Further, the result of the third-generation predicted by the first and second-generations is ‘2:1:0.5:0.5’ with ‘Akita:Chow-Chow:Jindo:Pungsan,’ respectively, and this individual has two ‘Akita,’ one ‘Chow-Chow,’ one ‘Jindo’ and ‘Pungsan’ 1:1 mix of the third-generation (grandparents).

Although this embodiment has been described focusing on topographical and external populations, it can be performed targeting all populations that can be divided into a diseased population, a control population, or a specific phenotype. If there is a data set composed of multiple disease populations and non-disease populations, it is possible to determine which disease populations are close to other samples that do not belong to the data set through this embodiment. Through this, it is possible to determine which disease a specific individual is more susceptible to. This may additionally provide and supplement the results with existing methods of measuring the risk of disease through specific biomarkers.

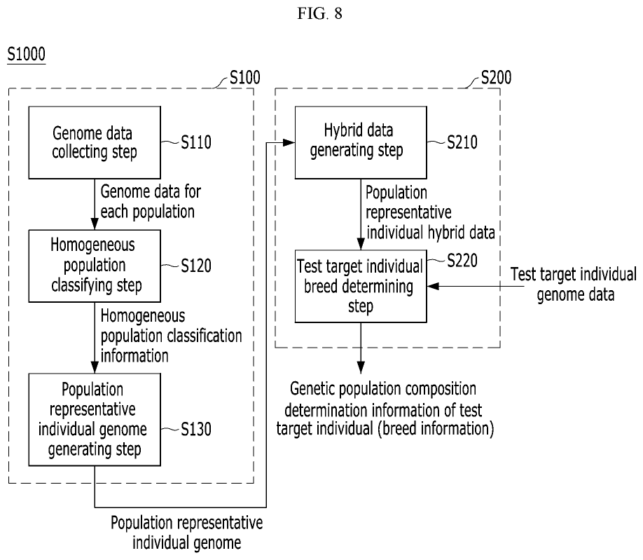

FIG. 8 is a flow chart showing the overall configuration of a method for determining a genetic population composition using specific reference genome data of populations and hybrids according to an embodiment of the present invention.

Referring to FIG. 8, the method for determining a genetic population composition using specific reference genome data of populations and hybrids S1000 according to another embodiment of the present invention may comprise at least one of a population representative individual selecting step S100 and a genetic population composition determining step S200.

The population representative individual selecting step S100 may measure the frequency of occurrence of a pre-selected genotype for individuals in the homogenous population and select a population representative individual for each of the homogenous populations according to the measured frequency of occurrence.

To this end, as shown in FIG. 8, the population representative individual selecting step S100 may comprise at least one of a genome data collecting step S110, a homogenous population classifying step S120, and a population representative individual genome generating step S130.

The genome data collecting step S110 may collect a large amount of genome data (sanger, NSG, micro-array, etc.) for each population (e.g., topographical and external populations) and store and manage the collected genome data for each population.

The homogenous population classifying step S120 may measure the genetic similarity between populations using a large amount of genome data collected through the genome data collecting step S110 and cluster and classify into homogenous populations according to the measurement result. The homogenous population classifying step S120 may cluster populations that can be classified as homogenous populations according to the degree of similarity for each population into homogenous populations and accumulate data accordingly. The homogenous population classifying step S120 may apply a method for measuring genetic similarity between individuals in a population, such as the ‘Admixture’ method shown in FIG. 2 and may not only identify and merge the same population, but also eliminate other individuals who are not members of the population through this method. If they are otherwise collected from different sources, they may be treated as different names of populations.

The population representative individual genome generating step S130 may measure the frequency of occurrence of a pre-selected genotype for each identical genetic location among individuals in the homogenous population and select the frequency of occurrence of a pre-selected genotype and select a population representative individual for each homogenous population according to the measured frequency of occurrence to create genomes for representative individuals of the population.

More specifically, the population representative individual genome generating step S130 may select the individual with the highest frequency of occurrence as the population representative individual of the first-generation in which a population representative individual may be selected in a random manner for two or more individuals having genotypes having the same frequency of occurrence. That is, in the process of generating a population representative through the measurement of the frequency of occurrence of a genotype for each genetic location among individuals in a population, for example, as shown in FIG. 3, a population representative may be randomly selected in the case of the same frequency of occurrence of genotype.

Further, when the frequency of occurrence is equal to or less than a predetermined reference frequency, the population representative individual genome generating step S130 may remove the corresponding individual. If there are heterogeneous individuals that have not been filtered through the homogenous population classifying step S120 by measuring the frequency of occurrence of the genotype, the genotype of the heterogeneous individual may be removed through the measurement of the frequency of occurrence of the genotype. For example, when a population consisting of 100 Koreans is collected, if one Japanese is not filtered out through the homogenous population classifying step S120, through the measurement of the frequency of occurrence of genotypes, genotypes commonly possessed by 99 Koreans are adopted, thereby minimizing the effect of one Japanese to the fullest.

Further, the population representative genome generating step S130 may measure the genetic similarity between population representative individuals in the same generation and select, if the similarity is higher than a predetermined reference level, the corresponding population representative individual as one common population representative individual. When the representative individual of the population first created in this embodiment is referred to as the first-generation population representative individual, the first-generation population representative individual refers to a collection of information about the structure and genotype of the genome that is frequently occurred in the population. If the first-generation representative of population A and the first-generation representative of population B are genetically very close, characteristics such as origin, traffic, common ancestry, and phenotype between populations A and B are identified to create common first-generation population representative.

The genetic population composition determining step S200 may generate hybrid data of the population representative individual for each generation through repetitive hybridization between the population representative individuals and determine the genetic population composition of the test target individual according to the genetic similarity between the hybrid data and the test target individual.

To this end, the genetic population composition determining step S200 may comprise at least one of a hybrid data generating step S210 and a test target individual breed determining step S220, as shown in FIG. 8.

The hybrid data generating step S210 may generate hybrid data of population representative individuals for each generation through repetitive hybridization between population representative individuals. FIG. 4 shows an example of a hybridization process, in which the genotype is determined according to Mendel's laws of inheritance in the hybridization process. The generated 1:1 hybrid is referred to as the second-generation, and genotype voting is performed on newly generated second-generation individuals to generate second-generation representative individuals as shown in FIG. 3. One second-generation representative individual includes the genetic information of the first-generation representative individual of the two populations in a 50:50 ratio. In this way, hybrid data for a 3rd generation individual can be created using the genetic information of the second-generation representative individual and the second-generation representative individual.

The hybrid data for the representative individual of each generation thus generated may also be used when generation representative individual data described later is generated. For example, as shown in FIG. 5, if population A, population B, and population C have a composition of 50:25:25, respectively, in a third-generation individual,‘A:B:C=50:25:25’ individual may be created using a first-generation population A representative and a second-generation population B-C representative. As in the case of creating second-generation representative individuals, the third-generation individuals may be created through repeated hybridization, and the third-generation representatives may be created using these third-generation individuals.

By the hybrid data generating step S210, the number of representatives up to the third-generation may be determined by Equation 2 below, which is a combination formula including duplication. That is, the combination in repetitive hybridization between population representative individuals according to the first-generation, second-generation, third-generation, and higher generations may be determined according to Equation 2 (Equation, #Representator) below.

[Equation 2]

| Generation | Equation | #Representator |
|---|---|---|
| 1^{st} | N | N |
| 2^{nd} | (n + 2 − 1)!/{(n − 1)!*2!} | (N − 1)!/2(n − 1)! − #(1^{st}) |
| 3^{rd} | (n + 4 − 1)!/{(n − 1)!*4!)} | (N + 3)!/24(N − 1)! − |
| | | #(2^{nd}) − #(1^{st}) |
| . . . | . . . | . . . |
| m | (n + 2\hat{ }(m − 1) − 1)!/{(n − | (N + 2\hat{ }(m − 1) − 1)!/{(N − |
| generation | 1)!*2\hat{ }(m − 1)!} | 1)!*2(m − 1)!} − Σ₁^{m-1} |
| | | # generations |

In Equation 2, Equation means the total number of population representative individuals of m generation without considering previous generations, #Representator means the total number of population representative individuals used in each generation, and N in Equation and #Representator means the number of populations. More specifically, Equation of Equation 2 represents the combination formula of populations including duplicates, n of Equation represents the number of populations to be identified, and m represents the number of generations. Equation represents the total number of population representations that may be had in m generation without considering the number of previous generations. N of #Representator represents the number of populations, and m represents the number of generations like Equation. Equation represents the total number of population representatives from each generation, and #Representator represents the number of population representatives directly used by each generation.

The test target individual breed determining step S220 may measure the genetic similarity between the hybrid data and the test target individual generated by the hybrid data generating step S210 and identify the breed of the test target individual (genetic population composition) according to the measurement result. More specifically, the test target individual breed determining step S220 may measure the genetic similarity with the test target individual among the hybrid data to assume the genetic population composition of the population representative individual corresponding to the hybrid data with the highest degree of similarity to the genetic population composition of the test target individual. In this way, which population is closest to a particular generation can be identified by comparing new individuals of the particular generation with representatives of that generation and representatives of previous generations.

For example, in order to determine which population is closest to the parent generation (second-generation), N's individual may be compared with the representatives of the first and second-generations to identify which representative is closest to ‘Identity-By-Descent.’ If it is closest to the representative A-B of the second-generation, the genetic population composition of N is represented by A-B, and if it is closest to the representative A of the first-generation, the population composition of N is represented by A-A. Although this embodiment describes the analysis method through the measurement of ‘Identity-By-Descent,’ it may include all other methods for measuring genetic similarity.

Further, the test target individual breed determining step S220 may sort the population representative individuals in the order of high genetic similarity with the test target individual, converts the genetic similarity of the respective sorted population representative individuals into a percentage, and divide the converted percentage value by the proportion of the respective population representatives in the total population representatives to estimate the divided value as an approximation of a positive integer, thereby identifying the genetic population composition of the test target individual of the next generation, not a specific generation. The test target individual breed determining step S220 may identify the next population or percentage through pattern analysis of genetic similarity results in order to confirm the percentage of the next generation and genetic population composition, not a specific generation. Here, pattern analysis may be performed by ensemble of several tests to confirm the result.

FIG. 6 shows a schematic view of how to predict the percentage of a population and the pedigree of a population in the next generation using the genetic similarity results for representatives of the first and second-generations. First of all, it is determined whether the input can be expressed with only the first and second-generations, and if it cannot be expressed with only the first and second-generations, as shown in FIG. 7, after sorting in order of similarity, a pattern for the result may be identified. The population according to the identified pattern is converted into a percentage and divided by the number of results in the next generation (the third-generation is the number of 4 representative individuals). As shown in FIG. 7, the results for ‘Akita,’ ‘Chow-Chow,’ ‘Jindo,’ and ‘Pungsan’ are converted into percentages (55%, 29%, 8%, and 8%), these values are divided by 0.25, i.e., each individual's share of the total population to estimate an approximate value (rounding method), thereby obtaining the third-generation result of ‘2, 1, 0.5, and 0.5,’ respectively, and the result may be estimated as the genetic population composition of the to-be-tested individual. Further, the result of the third-generation predicted by the first and second-generations is ‘2:1:0.5:0.5’ with ‘Akita:Chow-Chow:Jindo:Pungsan,’ respectively, and this individual has two ‘Akita,’ one ‘Chow-Chow,’ one ‘Jindo’ and ‘Pungsan’ 1:1 mix of the third-generation (grandparents).

Hereinafter, experimental examples of the system and method for determining genetic population composition using the specific reference genome data of populations and hybrids of the present invention are described.

A dog breed discrimination analysis was performed using the methodology according to this embodiment. A total of 8,344 dogs of more than 200 breeds (populations) were collected, and when using the method shown in Table 1 and FIG. 2 below, and only breeds registered with the Kennel Club in England, 6,799 dogs of 129 breeds (populations) of was applied to this experimental example.

TABLE 1

| Data set | # Dog | # SNPs |
|---|---|---|
| GEO | 2344 | 136,680 |
| WGS | 722 | 91,245,020 |
| Jorunal data | 237 | 119,035 |
| 6k Dog | 5,406 | 166,171 |
| Korean Dog | 192 | 173,662 |
| Sample and marker QC | 8,344 | 113,750 |

To test the created population representatives, a 7:3 Training Test division was performed as shown in Table 2 below.

TABLE 2

| No. | Breed | Train | Test |
|---|---|---|---|
| 1 | Afghan-Hound | 16 | 6 |
| 2 | Airedale-Terrier | 14 | 6 |
| 3 | Akita | 16 | 6 |
| 4 | Alaskan-Malamute | 17 | 7 |
| 5 | Australin-Cattle-Dog | 11 | 4 |
| 6 | Australian Shepherd | 21 | 9 |
| 7 | Australian-Terrier | 8 | 3 |
| 8 | Basenji | 31 | 12 |
| 9 | Basset-Hound | 21 | 9 |
| 10 | Beagle | 33 | 13 |
| 11 | Bearded-Collie | 8 | 3 |
| 12 | Belgian-Malinois | 8 | 3 |
| 13 | Belgian-Tervuren | 28 | 12 |
| 14 | Bergamasco | 7 | 3 |
| 15 | Bernese-Mountain-Dog | 41 | 17 |
| 16 | Bichon-Frise | 16 | 6 |
| 17 | Bloodhound | 12 | 5 |
| 18 | Bolognese | 14 | 5 |
| 19 | Border-Collie | 64 | 27 |
| 20 | Border-Terrier | 30 | 12 |
| 21 | Borzoi | 16 | 6 |
| 22 | Boston-Terrier | 22 | 9 |
| 23 | Bouvier-Des-Flandres | 8 | 3 |
| 24 | Boxer | 118 | 50 |
| 25 | Bracco-Italiano | 7 | 2 |
| 26 | Briard | 8 | 3 |
| 27 | Brittany | 21 | 9 |
| 28 | Bullmastiff | 16 | 6 |
| 29 | Bull-Terrier | 41 | 17 |
| 30 | Cairn-Terrier | 83 | 35 |
| 31 | Canaan-Dog | 9 | 3 |
| 32 | Cardigan-Welsh-Corgi | 10 | 4 |
| 33 | Catahoula-Leopard-Dog | 7 | 3 |
| 34 | Cavalier-King-Charles-Spaniel | 109 | 46 |
| 35 | Chesapeake-Bay-Retriever | 12 | 4 |
| 36 | Chihuahua | 19 | 7 |
| 37 | Chinese-Crested | 11 | 4 |
| 38 | Chow-Chow | 13 | 5 |
| 39 | Cirneco-dell-Etna | 7 | 2 |
| 40 | Cocker-Spaniel | 50 | 21 |
| 41 | Collie | 14 | 6 |
| 42 | Dachshund | 39 | 16 |
| 43 | Dalmatian | 18 | 7 |
| 44 | Doberman-Pinscher | 45 | 18 |
| 45 | Doque-de-Bordeaux | 7 | 2 |
| 46 | English-Bulldog | 25 | 10 |
| 47 | English-Cocker-Spaniel | 29 | 12 |
| 48 | English-Setter | 70 | 30 |
| 49 | English-Springer-Spaniel | 83 | 35 |
| 50 | Entlebucher-Sennenhund | 6 | 2 |
| 51 | Eurasier | 10 | 4 |
| 52 | Finnish-Spitz | 9 | 3 |
| 53 | Flat-Coated-Retriever | 11 | 4 |
| 54 | Foxhound | 7 | 3 |
| 55 | Fox-Terrier-Wire | 9 | 3 |
| 56 | French-Bulldog | 28 | 11 |
| 57 | German-Shepherd-Dog | 217 | 92 |
| 58 | German-Shorthaired-Pointer | 16 | 6 |
| 59 | Giant-Schnauzer | 12 | 4 |
| 60 | Glen-of-Imaal-Terrier | 7 | 2 |
| 61 | Golden-Retriever | 234 | 99 |
| 62 | Gordon-Setter | 33 | 14 |
| 63 | Great-Dane | 38 | 15 |
| 64 | Greater-Swiss-Mountain-Dog | 14 | 5 |
| 65 | Greenland-Sledge-Dog | 9 | 3 |
| 66 | greyhound | 220 | 94 |
| 67 | Grey-Wolf | 33 | 13 |
| 68 | Griffon-Bruxellois | 54 | 23 |
| 69 | Havanese | 38 | 16 |
| 70 | Ibizan-Hound | 7 | 2 |
| 71 | Irish-Setter | 14 | 6 |
| 72 | Irish-Water-Spaniel | 10 | 3 |
| 73 | Irish-Wolfhound | 290 | 123 |
| 74 | Italian-Greyhound | 23 | 9 |
| 75 | Jack-Russell-Terrier | 29 | 12 |
| 76 | Jindo | 80 | 34 |
| 77 | Keeshound | 15 | 6 |
| 78 | Kuvasz | 7 | 3 |
| 79 | Labrador-Retriever | 527 | 225 |
| 80 | Lagotto-Romagnolo | 17 | 7 |
| 81 | Leonberger | 17 | 7 |
| 82 | Lhasa-Apso | 11 | 4 |
| 83 | Maltese | 61 | 26 |
| 84 | Maremma | 10 | 4 |
| 85 | Mastiff | 23 | 9 |
| 86 | Miniature-Pinscher | 20 | 8 |
| 87 | Miniature-Schnauzer | 49 | 20 |
| 88 | Neapolitan-Mastiff | 17 | 7 |
| 89 | Newfoundland | 81 | 34 |
| 90 | Norfolk-Terrier | 16 | 6 |
| 91 | Norwich-Terrier | 12 | 5 |
| 92 | Nova-Scotia-Duck-Tolling-Retriever | 25 | 10 |
| 93 | Old-English-Sheepdog | 14 | 5 |
| 94 | Otterhound | 9 | 3 |
| 95 | Papillon | 29 | 12 |
| 96 | Pekingese | 17 | 6 |
| 97 | Pembroke-Welsh-Corgi | 49 | 21 |
| 98 | Petit-Basset-Griffon-Vendeen | 8 | 3 |
| 99 | Pomeranian | 19 | 8 |
| 100 | Portuguese-Water-Dog | 22 | 9 |
| 101 | Pug | 21 | 9 |
| 102 | Rat-Terriel | 8 | 3 |
| 103 | Rhodesian-Ridgeback | 11 | 4 |
| 104 | Rottweiler | 175 | 74 |
| 105 | Saint-Bernard | 32 | 13 |
| 106 | Saluki | 21 | 8 |
| 107 | Samoyed | 16 | 6 |
| 108 | Schipperke | 19 | 7 |
| 109 | Scottish-Deerhound | 9 | 3 |
| 110 | Scottish-Terrier | 25 | 10 |
| 111 | Shar-pei | 22 | 9 |
| 112 | Shetland-Sheepdog | 27 | 11 |
| 113 | Shiba-Inu | 7 | 3 |
| 114 | Shih-Tzu | 27 | 11 |
| 115 | Siberian-Husky | 21 | 9 |
| 116 | Soft-Coated-Wheaten-Terrier | 13 | 5 |
| 117 | Spinone-Italiano | 13 | 5 |
| 118 | Staffordshire-Bull-Terrier | 17 | 6 |
| 119 | Standard-Poodle | 10 | 4 |
| 120 | Standard-Schnauzer | 14 | 5 |
| 121 | Tibetan-Mastiff | 13 | 5 |
| 122 | Tibetan-Spaniel | 26 | 10 |
| 123 | Tibetan-Terrier | 11 | 4 |
| 124 | TM-Poodle | 32 | 13 |
| 125 | Vizsla | 71 | 30 |
| 126 | Weimaraner | 26 | 11 |
| 127 | West-Highland-White-Terrier | 28 | 12 |
| 128 | Whippet | 12 | 5 |
| 129 | Yorkshire-Terrier | 200 | 85 |

| | | |
|---|---|---|
| Greater-Swiss-Mountain-Dog | 4793 | 1975 |

6769

Referring to Table 2, the animals were divided into the training set of 4,793 animals and the training of 1,976 animals, and since the number of data for each breed is different, the ratio was adjusted to 7:3 in consideration of this.

Further, as shown in FIG. 3, 129 population representative reference genomes were generated through genotype voting, a large amount of second-generation population was generated using Mendel's genetic law shown in FIG. 4, and representative reference genomes of second-generation hybrids of 8,256 animals were made through voting again by the method shown in FIG. 3.

As such, about 12 million were generated as a representative standard genome of the third-generation hybrid through the method shown in FIGS. 3, 4, and 5. The number of representative genome data created is shown in Table 3 below, which is a number according to Equations 1 and 2.

TABLE 3

| | | #Breed |
|---|---|---|
| Generation | #Representator | Representator |
| 1^{st} | N | 129 |
| 2^{nd} | (N − 1)!/2(n − 1)! − #(1^{st}) | 8,256 |
| 3^{rd} | (N + 3)!/24(N − 1)! − #(2^{nd}) − #(1^{st}) | 12,074,400 |

In order to produce hybrid data for other tests in this example, the test data set of 1976 was randomly crossed, as shown in Table 1, 500 animals were made in a combination ratio of 50:50, 25:25:25:25, 75:25, and 50:25:25, respectively. The test was conducted with a total of 3,976 test data, including 1,976 purebreds and 2,000 hybrids created through simulation. The fourth-generation composition breeds were identified through the method shown in FIGS. 6 and 7, and the results are shown in Table 1.

Here, the singularity is that in order to compare up to the third-generation, about 12 million similarity measurement tests must be performed, but the first and second-generation tests were conducted first (8,385 times), the two closest breeds were fixed, and the third-generation test was performed. Therefore, the number of test runs was 8,385+8,385, and a total of 16,770 (0.14%) comparisons were performed.

The Jack Russell Terrier, which is a hybrid of many breeds, exhibits characteristics that are genetically similar to many breeds. To adjust these characteristics, one term was added in the combination test shown in FIG. 6 to adjust the effect of the Jack Russell Terrier.

According to this experimental example, 4,000 dogs of 129 breeds were created through the population representative individual generation unit, through another about 4,000 dogs (1,976 were generated by actual genome data and 2,000 were generated by ‘simulated mix data’), the fourth-generation population composition was identified through the genetic population composition determination unit, and as a result of the fourth-generation conversion, the true positive rate (TPR) was 93.4% on average, resulting in compliance.

Hereinafter, a comparative example of the above-described experimental example is described.

‘Labradodle’ is a hybrid of ‘Labrado-Retriver’ and Poodle. In Table 4 below, it can be seen how the breed composition is matched when the genome data of ‘Labradoodle’ is applied to the system and method of this embodiment.

TABLE 4

| Data source | Individual ID | Breed | Results |
|---|---|---|---|
| Pilot, Małgorzata, et al. | 1320 | Labradoodle | Jack-Russell-Terrier: 25%, |
| Proceedings of the Royal | | | Standard-Poodle: 25%, |
| Society B: Biological | | | Labrador-Retriever: 25%, |
| Sciences 282.1820 (2015): | | | *TM-Poodle: 25% |
| 20152189. | 1344 | Labradoodle | Labrador-Retriever: 48.3%, |
| | | | *TM-Poodle: 47.0%, |
| | | | Brittany: 4.7% |
| | 582 | Labradoodle | Standard-Poodle: 64.1%, |
| | | | Labrador-Retriever: 35.9% |
| | 4566 | Miniature- | Labrador-Retriever: 57.3%, |
| | | Labradoodle | *TM-Poodle: 42.7% |
| | 570 | Miniature- | *TM-Poodle: 48.3%, |
| | | Labradoodle | Labrador-Retriever: 42.1%, |
| | | | Standard-Poodle: 9.6% |

*TM-Poodle: Toy and Miniature Poodle

‘Cane-Corso’ is a breed that does not exist in the reference genome used in the present invention (see Table 2). However, it is possible to identify which breed is made up of a combination of which breeds. n fact, according to the ‘American Kennel Club’, it is specified as the closest breed to ‘Neapolitan Mastiff’, and as shown in Table 5 below, the most combinations of ‘Neapolitan-Mastiff’ and other breeds can be obtained.

TABLE 5

| Data source | Individual ID | Results |
|---|---|---|
| GEO, WGS | CaneCorso01 | Neapolitan-Mastiff: 59.8%, Staffordshire-Bull-Terrier: 32.3%, Bullmastiff: 4.6%, Mastiff: 3.2% |
| | GSM2196600 | Neapolitan-Mastiff: 66.8%, Bullmastiff: 30.5%, Staffordshire-Bull-Terrier: 2.7% |
| | GSM2196602 | Neapolitan-Mastiff: 56.0%, Rottweiler: 24.0%, |
| | | Staffordshire-Bull-Terrier: 11.0%, English-Bulldog: 6.0%, Bullmastiff: 3.0% |
| | GSM3424513 | Neapolitan-Mastiff: 61.5%, Bullmastiff: 30.0%, |
| | | Boxer: 5.6%, Boston-Terrier: 2.9% |
| | GSM3424514 | Neapolitan-Mastiff: 49.1%, Bullmastiff: 32.7%, |
| | | Staffordshire-Bull-Terrier: 4.4%, Boxer: 11.0%, Boston-Terrier: 2.8% |
| | GSM3424515 | Ibizan-Hound: 25%, Staffordshire-Bull-Terrier: 25%, |
| | | Bullmastiff: 25%, Neapolitan-Mastiff: 25% |
| | GSM3502211 | Jack-Russell-Terrier: 8.7%, Neapolitan-Mastiff: 48.2%, |
| | | Boxer: 27.9%, Staffordshire-Bull-Terrier: 2.5%, |
| | | Bullmastiff: 7.2%, Mastiff: 5.5% |
| | GSM3502218 | Neapolitan-Mastiff: 57.8%, Staffordshire-Bull-Terrier: 30.8%, |
| | | Bullmastiff: 4.8%, Boxer: 3.1%, Saint-Bernard: 3.5% |
| | PFZ14C09 | Rottweiler: 48.6%, Neapolitan-Mastiff: 42.4%, |
| | | Staffordshire-Bull-Terrier: 3.6%, Great-Dane: 5.4% |
| | PFZ15B09 | Neapolitan-Mastiff: 57.1%, Jack-Russell-Terrier: 32.0%, |
| | | English-Bulldog: 3.5%, Bullmastiff: 4.3%, Rat-Terrier: 3.1% |
| | PFZ15D04 | Neapolitan-Mastiff: 55.8%, Rottweiler: 38.9%, Staffordshire-Bull-Terrier: 5.4% |
| | PFZ15F02 | Neapolitan-Mastiff: 48.8%, Rottweiler: 37.4%, |
| | | Boxer: 6.8%, French-Bulldog: 3.5%, Boston-Terrier: 3.5% |
| | PFZ15H07 | Neapolitan-Mastiff: 52.6%, Rottweiler: 35.5%, |
| | | Boxer: 2.6%, English-Bulldog: 6.0%, Bullmastiff: 3.3% |
| | PFZ42B04 | Boxer: 25%, Staffordshire-Bull-Terrier: 25%, Neapolitan-Mastiff: 25%, |
| | | Rottweiler: 25% |
| | PFZ42H05 | Neapolitan-Mastiff: 60.8%, Rottweiler: 34.1%, Staffordshire-Bull-Terrier: 5.2% |

In the present invention, it is possible to preserve genetic characteristics through the generation of representative individuals. For example, when creating a specific cancer representative genome in a population that includes Koreans, Japanese, and British, differences in many genetic loci will be observed due to regional differences. However, when approaching the concept of conservation of genetic common parts, the diversity of the population disappears and a specific genetic locus can be extracted.

1. A system for generating specific reference genetic data of mixture of population and disease population or breed or hybrid and determining genetic population composition, the system comprising:
a population representative individual selection unit for measuring the frequency of occurrence of preselected genotypes for individuals in homogenous populations and selecting population representative individuals for the respective homogenous populations according to the measured frequency of occurrence; and
a genetic population composition determination unit for generating hybrid data of the population representative individuals for the respective generation through repetitive hybridization between the population representative individuals and determining the genetic population composition of the test target individual according to the genetic similarity between the hybrid data and the test target individual.

2. The system of claim 1, wherein the population representative individual selection unit comprises:
a genome data collection unit for collecting genomic data for the respective populations;
a homogenous population classification unit for measuring genetic similarity between populations using the genetic data and classifying into homogenous populations according to the measurement result; and
a population representative individual genome generation unit for measuring the frequency of occurrence of a preselected genotype at the same genetic locus among individuals in the homogenous population and selecting population representative individuals for the respective homogenous populations according to the measured frequency of occurrence to generate a genome for the selected population representative individuals.

3. The system of claim 2, wherein the homogenous population classification unit removes individuals that do not cluster into homogenous populations.

4. The system of claim 2, wherein the population representative individual generation unit selects an individual with the highest frequency of occurrence as the population representative individual, wherein the population representative individual is randomly selected from two or more individuals having the genotype with the same frequency of occurrence.

5. The system of claim 2, wherein the population representative individual generation unit removes the individual when the frequency of occurrence is less than or equal to a predetermined cutoff frequency.

6. The system of claim 2, wherein the population representative individual generation unit measures the genetic similarity between the population representative individuals within the same generation and selects the population representative individual as one common population representative individual when the measured similarity is higher than a predetermined cutoff similarity.

7. The system of claim 1, wherein the genetic population composition determination unit comprises:
a hybrid data generation unit for generating hybrid data of the population representative individuals for the respective generations through repetitive hybridization between the population representative individuals; and
a test target individual breed determination unit for measuring the genetic similarity between the hybrid data and the test target individual and determining the breed of the test target individual according to the measurement result.

8. The system of claim 7, wherein the hybrid data generation unit determines a combination according to the formula (Equation, #Representator) at the time of repeated hybridization between the first, second, third and higher generation population representative individuals,

| Generation | Equation | #Representator |
|---|---|---|
| 1^{st} | N | N |
| 2^{nd} | (n + 2 − 1)!/{(n − 1)!*2!} | (N − 1)!/2(n − 1)! − #(1^{st}) |
| 3^{rd} | (n + 4 − 1)!/{(n − 1)!*4!)} | (N + 3)!/24(N − 1)! − |
| | | #(2^{nd}) − #(1^{st}) |
| . . . | . . . | . . . |
| m | (n + 2\hat{ }(m − 1) − 1)!/{(n − | (N + 2\hat{ }(m − 1) − 1)!/{(N − |
| generation | 1)!*2\hat{ }(m − 1)!} | 1)!*2(m − 1)!} − Σ₁^{m-1} |
| | | # generations |

wherein the Equation represents the total number of population representative individuals that m generation has without considering previous generations,
wherein the #Representator represents the total number of population representative individuals used in the respective generations, and
wherein N of the Equation and #Representator is the number of populations.

9. The system of claim 7, wherein the test target individual breed determination unit presumes that the genetic population composition of the population representative individual corresponding to the hybrid data having the highest genetic similarity with the test target individual among the hybrid data is the genetic population composition of the test target individual.

10. The system of claim 7, wherein the test target individual breed determination unit sorts the population representative individuals in the order of high genetic similarity with the test target individual, converts the genetic similarity of the respective sorted population representative individuals into a percentage, and divides the converted percentage value by the proportion of the respective population representatives in the total population representatives to estimate the divided value as an approximation of a positive integer, thereby identifying the genetic population composition of the test target individual of the next generation, not a specific generation.

11. A method for generating specific reference genetic data of mixture of population and disease population or breed or hybrid and determining genetic population composition, the method comprising:
a population representative individual selecting step for measuring the frequency of occurrence of preselected genotypes for individuals in homogenous populations and selecting population representative individuals for the respective homogenous populations according to the measured frequency of occurrence; and
a genetic population composition determining step for generating hybrid data of the population representative individuals for the respective generation through repetitive hybridization between the population representative individuals and determining the genetic population composition of the test target individual according to the genetic similarity between the hybrid data and the test target individual.

12. The method of claim 11, wherein the population representative individual selecting step comprises:
a genome data collecting step for collecting genomic data for the respective populations;
a homogenous population classifying step for measuring genetic similarity between populations using the genetic data and classifying into homogenous populations according to the measurement result; and
a population representative individual genome generating step for measuring the frequency of occurrence of a preselected genotype at the same genetic locus among individuals in the homogenous population and selecting population representative individuals for the respective homogenous populations according to the measured frequency of occurrence to generate a genome for the selected population representative individuals.

13. The method of claim 12, wherein the homogenous population classifying step removes the individual that does not cluster into homogenous populations.

14. The method of claim 12, wherein the population representative individual genome generating step selects an individual with the highest frequency of occurrence as the population representative individual, wherein the population representative individual is randomly selected from two or more individuals having the genotype with the same frequency of occurrence.

15. The method of claim 12, wherein the population representative individual genome generating step removes the individual when the frequency of occurrence is less than or equal to a predetermined cutoff frequency.

16. The method of claim 12, wherein the population representative individual genome generating step measures the genetic similarity between the population representative individuals within the same generation and selects the population representative individual as one common population representative individual when the measured similarity is higher than a predetermined cutoff similarity.

17. The method of claim 11, wherein the genetic population composition determining step comprises:
a hybrid data generating step for generating hybrid data of the population representative individuals for the respective generations through repetitive hybridization between the population representative individuals; and
a test target individual breed determining step for measuring the genetic similarity between the hybrid data and the test target individual and determining the breed of the test target individual according to the measurement result.

18. The method of claim 17, wherein the hybrid data generating step determines a combination according to the formula (Equation, #Representator) at the time of repeated hybridization between the first, second, third and higher generation population representative individuals,

| Generation | Equation | #Representator |
|---|---|---|
| 1^{st} | N | N |
| 2^{nd} | (n + 2 − 1)!/{(n − 1)!*2!} | (N − 1)!/2(n − 1)! − #(1^{st}) |
| 3^{rd} | (n + 4 − 1)!/{(n − 1)!*4!)} | (N + 3)!/24(N − 1)! − |
| | | #(2^{nd}) − #(1^{st}) |
| . . . | . . . | . . . |
| m | (n + 2\hat{ }(m − 1) − 1)!/{(n − | (N + 2\hat{ }(m − 1) − 1)!/{(N − |
| generation | 1)!*2\hat{ }(m − 1)!} | 1)!*2(m − 1)!} − Σ₁^{m-1} |
| | | # generations |

wherein the Equation represents the total number of population representative individuals that m generation has without considering previous generations,
wherein the #Representator represents the total number of population representative individuals used in the respective generations, and
wherein N of the Equation and #Representator is the number of populations.

19. The method of claim 17, wherein the test target individual breed determining step presumes that the genetic population composition of the population representative individual corresponding to the hybrid data having the highest genetic similarity with the test target individual among the hybrid data is the genetic population composition of the test target individual.

20. The method of claim 17, wherein the test target individual breed determining step sorts the population representative individuals in the order of high genetic similarity with the test target individual, converts the genetic similarity of the respective sorted population representative individuals into a percentage, and divides the converted percentage value by the proportion of the respective population representatives in the total population representatives to estimate the divided value as an approximation of a positive integer, thereby identifying the genetic population composition of the test target individual of the next generation, not a specific generation.
